(19) United States
(12) Patent Application Publication
(10) Pub. No.: US 2011/0172671 A1
(43) Pub. Date: Jul. 14, 2011

(54) Orthopedic/Spine Bone Mill

(76) Inventors: Michael Mcmillan, Saint-Lazare (CA); Ariel Dujovne, Montreal (CA); Marc-Andre Thebault, Anjou (CA); Cameron M.L. Clokie, Toronto (CA)

(21) Appl. No.: 13/119,747

(22) PCT Filed: Oct. 9, 2009
(86) PCT No.: PCT/CA2009/001417

§ 371 Date: Mar. 18, 2011

Related U.S. Application Data
(60) Provisional application No. 61/104,112, filed on Oct. 9, 2008.

(51) Int. Cl. A61B 17/16

(52) U.S. Cl. 606/83

(57) ABSTRACT

A orthopedic/spine bone mill comprising a vessel and a rotatable grinding tool to produce ground autologous and or allograft bone particles for surgical bone reconstruction use, wherein the vessel includes a wall defining a circular cylindrical chamber having a concentric axis. The vessel further includes generally concave or a cup shaped bottom wall which corresponds to the shape of the cutting blade and a lid adapted to be removably connected to the vessel. The lid includes an opening coincident with the axis. The grinding tool includes an elongated shaft extending through the opening in the Jid along the axis with a cutting blade mounted to the shaft within the chamber whereby the cutting blade can be rotated by motive device, such as a drill find in the operating room, engaging the shaft exterior of the vessel to cut bone, placed in the vessel, into said bone particles.

### FIELD OF THE INVENTION

The present invention relates to a bone grinding apparatus and, more particularly, to a portable bone mill for use in autologous and/or autograft bone grafts.

### BACKGROUND OF THE INVENTION

Bone grinders or bone mills have been developed in the recent past to allow surgeons, particularly when dealing with oral/maxillofacial, orthopedic, periodontal, and implant applications, to perform autologous bone grafts using autogenous bone from local reservoirs. Such bone mills allow the patient to have his or her own bone particles implanted when there is a preference to using autograft to address concerns over the possibility of rejection or infection. For instance, in an oral/maxillofacial intervention, the surgeon can use bone from the patient's mandibular symphsis or ramus, then grind the bone with the bone mill, and then utilize the bone particles to repair small bone defects and to achieve bone augmentation. Such procedures reduce the costs of surgery compared where other products, such as HA granules, processed coral, or freeze-dried bone are used.

U.S. Pat. No. 5,769,853 (Quetin) issued Jun. 23, 1998, U.S. Pat. No. 5,918,821 (Grooms et al.), issued Jul. 6, 1999, and U.S. Pat. No. 6,287,312 (Clokie et al.) issued Sep. 11, 2001 are representative of bone grinders or mills which can produce small bone particles for the purpose of autologous bone grafts.

In light of the existing bone mills as represented by these patents and others available in the marketplace, there is a need for a simple, power-driven bone mill which can be easily utilized in a surgical environment, using the power tools that are within the current repertory of surgeons in the field.

The present invention seeks to meet this and related needs.

### SUMMARY OF THE INVENTION

It is an aim of the present invention to provide a bone mill having a simple construction and adapted to be operated by available power tools in hospital operating rooms.

It is a further aim of the present invention to provide a hygienic bone mill that has a simple design and that can be used with a one-use milling or cutting blade which can be easily replaced.

It is a further aim of the present invention to provide a portable bone mill which can be hand-held or clamped on a flat surface during operation.

An orthopedic/spine bone mill in accordance with the present invention comprises a vessel and a rotatable grinding tool to produce ground autologous and/or autograft bone particles for surgical bone reconstruction use, wherein the vessel includes a wall-defining a circular cylindrical chamber having a concentric axis, the vessel further includes a cup shaped bottom wall which corresponds to the shape of the cutting blade, a lid adapted to be removably connected to the vessel, the lid including an opening coincident with the axis, the grinding tool including an elongated shaft extending through the opening in the lid along the axis, a cutting blade mounted to an end of the shaft within the chamber whereby the cutting blade can be rotated by motive means engaging the shaft, exterior of the vessel, to cut bone, placed in the vessel, into said bone particles.

In a representative embodiment of the present invention, a bone mill comprises a vessel and a rotatable grinding tool to produce ground autologous and/or autograft bone particles for surgical bone reconstruction use, wherein the vessel includes a wall defining a generally circular cylindrical chamber having a concentric axis, the vessel further includes a cup shaped bottom wall which corresponds to the shape of the cutting blade, a lid adapted to be removably connected to the vessel, the lid including an opening coincident with the axis, the grinding tool including an elongated shaft extending through the opening in the lid along the axis, the lid including bearing means for the shaft of the grinding tool to permit the shaft to rotate on the axis, a cutting blade mounted to an end of the shaft within the chamber and a bone gripping plate on the bottom wall whereby the cutting blade can be rotated by motive means engaging the shaft, exterior of the vessel, to cut bone, placed in the vessel against the bone gripping plate, into said bone particles.

A suitable blade design is one in the form of a thin plate having rasp openings defined by rasp cutting edges for cutting the bone against the gripping plate and passing bone particles through the rasp openings in the cutting blade within the chamber opposite to the gripping plate.

Optionally and advantageously, the bone mill may be utilized with single use cutting blades which are removably mounted to a blade support mounted to the end of the shaft within the vessel, and a blade retention member extends through a central opening in the blade and engages the support means. It is contemplated that the cutting blade may also be fixed to the shaft. The entire grinding tool may be manufactured so as to be sold as a disposable, single use tool.

Other objects, advantages and features of the present invention will become apparent upon reading of the following non-restrictive description of embodiments thereof, given by way of example only with reference to the accompanying drawings.

### BRIEF DESCRIPTION OF THE DRAWINGS

Having thus generally described the nature of the invention, reference will now be made to the accompanying drawings, showing by way of illustration, a representative embodiment thereof, and in which:

FIG. 1 is an assembled perspective view showing an orthopedic/spine bone mill in accordance with the present invention;

FIG. 2 shows assembled views (A and C) and disassembled views (B and D) of a representative blade assembly of the embodiment of the bone mill of FIG. 1;

FIG. 3 reveals cross-sectional views (E, F and G) of the embodiment of the bone mill of FIG. 1;

FIG. 4 illustrates a side view (H) and a top view (I) of the lid of the embodiment of the bone mill of FIG. 1; and

FIG. 5 is a representative commercial embodiment of the bone mill shown in FIG. 1.

### DETAILED DESCRIPTION OF THE INVENTION

As used herein, the term the term “concave” means curved inward, like the inside of a circle or sphere.

As used herein, the “motive means” refers to a device that imparts a circular motion and that is suitable for use with the present invention. An example would be a hand drill.

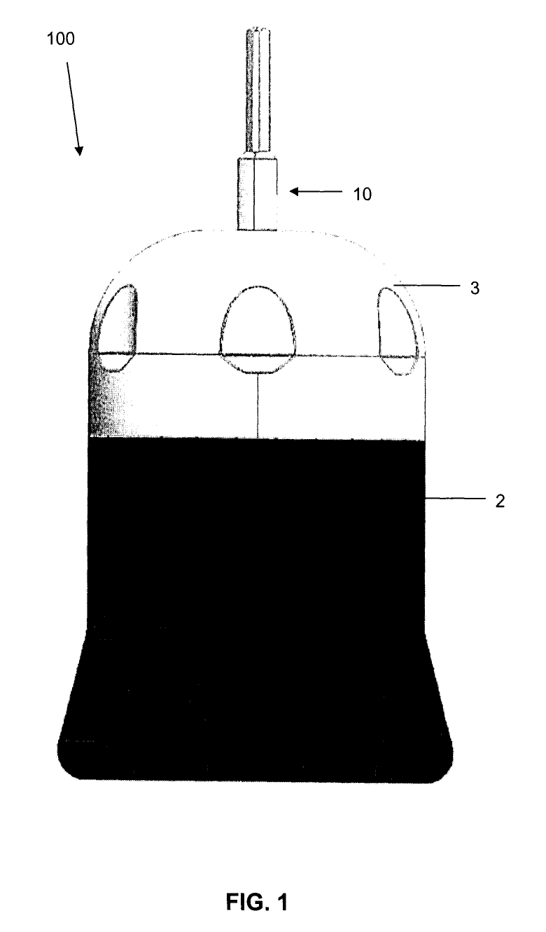

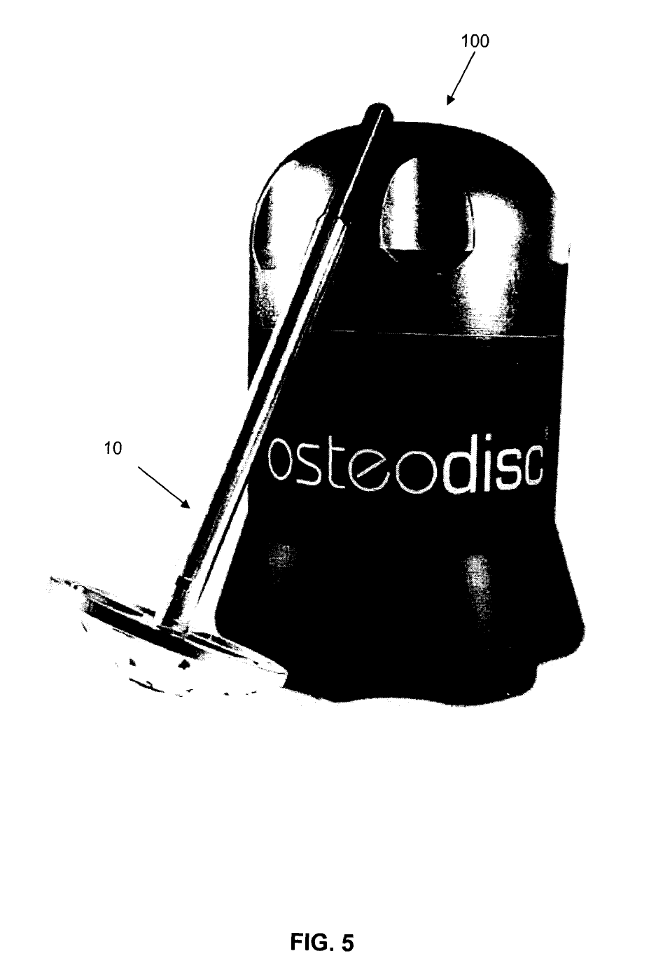

Referring to FIGS. 1 through 5, an orthopedic/spine bone mill in accordance with the present invention is shown is generally designated by the numeral 100 and includes a vessel 2 and a lid 3. A grinding tool 10 extends through the lid 3 into the vessel 2 (FIG. 1).

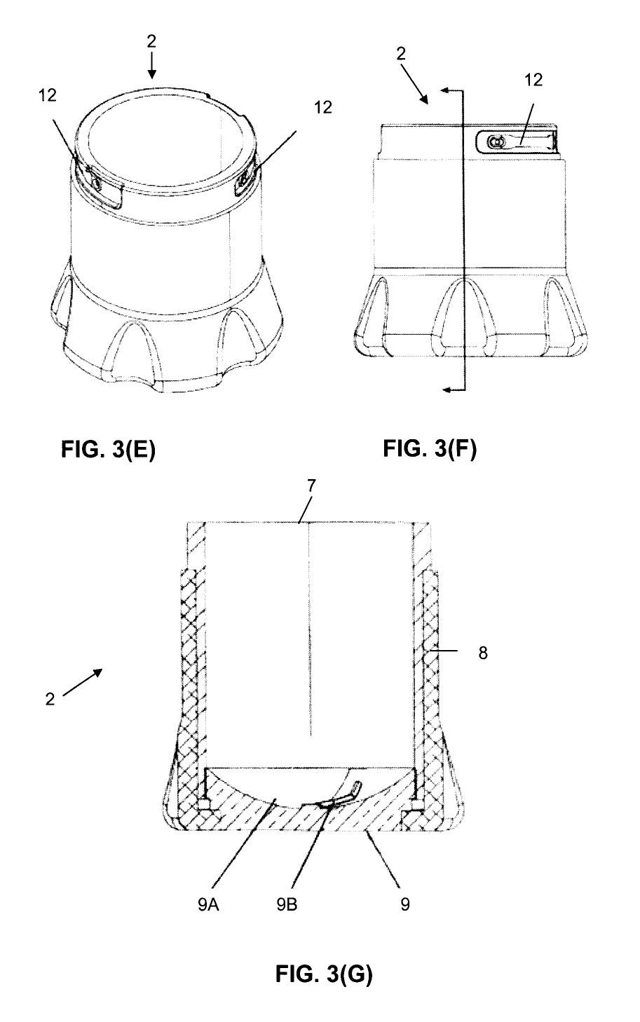

A shown in FIG. 3, the vessel 2 includes a base 9 with a generally concave or cup shaped surface 9A. A cylindrical wall extends from the base 9 and defines a cylindrical chamber 7 also being generally concave or cup shaped. The cylindrical wall includes a threaded segment with leafsprings 12 adapted to receive the lid having a mating, threaded, cylindrical wall portion.

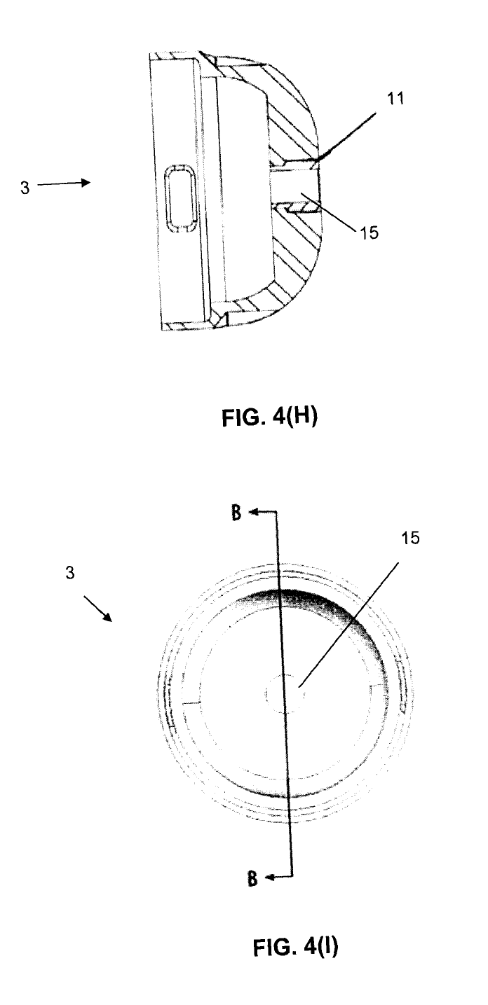

As shown in FIG. 4, the lid 3 includes a bore 15 to accommodate a bushing sleeve 11 which is threaded and further held in place with a suitable adhesive, such as Loctite®.

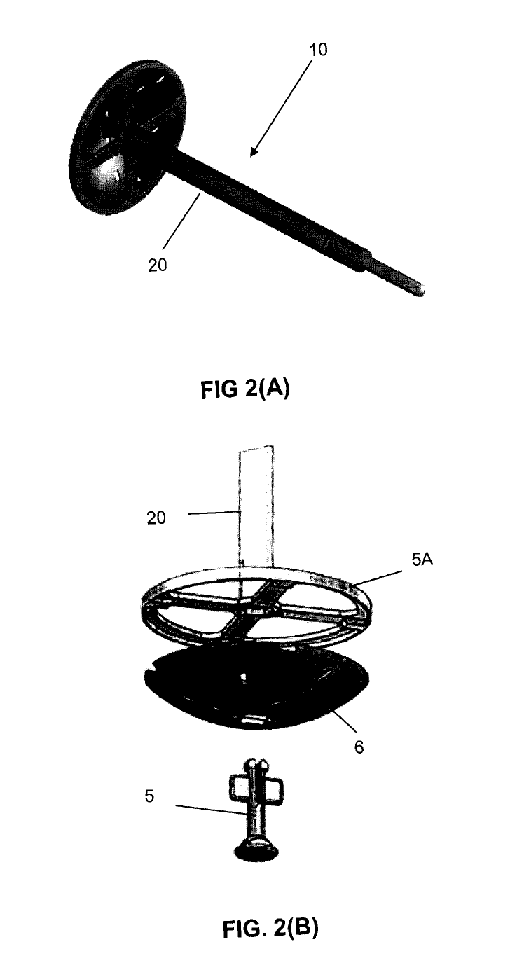

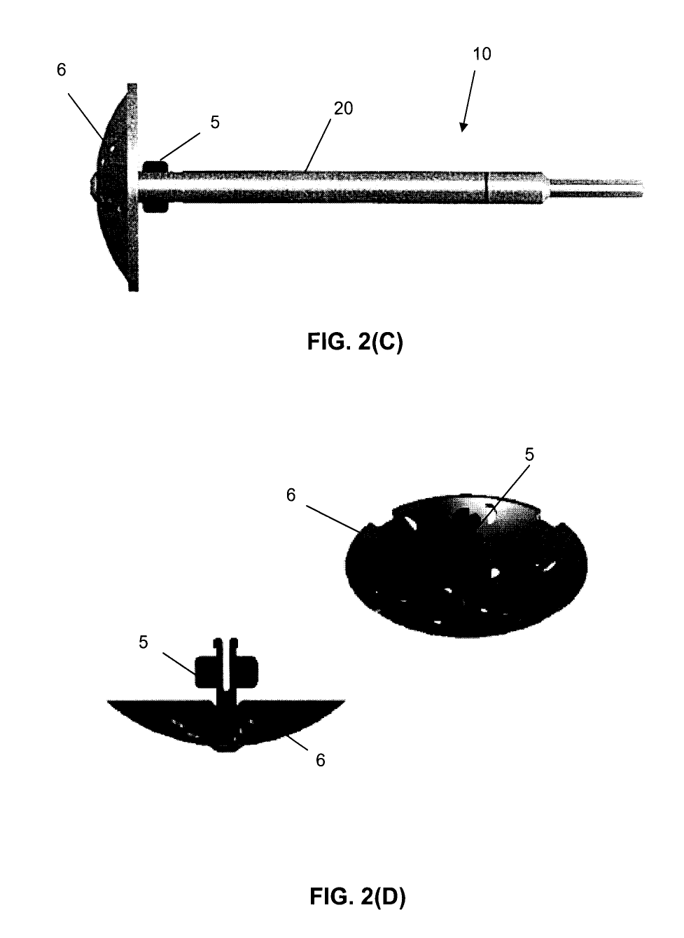

FIG. 2 reveals the details of the grinding tool 10. FIGS. 2(A) and 2(C) show top and side perspective views, respectively, of the grinding tool 10, while FIGS. 2(B) and 2(C) illustrate the finer elements of the blade 6 and blade support 5A. The shaft 20 of the grinding tool 10 includes a portion adapted to fit for rotational movement in the bore 15 of the lid 3 against bushing sleeve 11. A blade retainer 5 comprises a hub which engages the center of the cutting blade 6 and locks into the shaft 20. In the particular embodiment shown, the blade support 5A has four spokes that extend radially from the center of the hub to the periphery where the hub's external part engages the blade to provide axial and rotational support to the blade. Similar arrangements are possible and are within the purview of one of skill in the art.

The cutting blade 6 is a generally concave or cup shaped thin blade having rasp openings similar to a cheese grater. The removable blade retainer 5 is a fastener having a head and a shank, whereby the shank is adapted to engage the shaft 20, and the head has an axial dimension which corresponds to the minimum spacing between the cutting blade 6 and the bowl gripping plate 9B during the operation of the bone mill 100. The cutting blade 6 may be made of stainless steel or any other suitable metal or alloy, as known in the art. The cutting blade 6 is a single-use blade and is easily removed from the blade support and is held there by the blade retainer 5.

The end opposite the blade end of shaft 20 is adapted to be engaged by any drill that is commonly used in operating rooms. The shaft 20 preferably would be rotated between approximately 500 and 3,000 rpm with a torque of up to 100 newton/m.

In the embodiment illustrated, the generally concave or cup shaped surface 9A, made of hard metal such as stainless steel, is irremovably inserted in a recess formed at the bottom of the chamber 7 wall. The bowl gripping plate 9B includes radially extending, uninterrupted ridges which are ramp-shaped so that the leading edge of the ramp counters the rotational direction of the grinding tool 10. The ridges help to retain the bone pieces while the grinding tool 10 is rotated.

In operation, pieces of freshly removed bone or allografts are placed within the vessel 2, in the chamber 7 on the cup shaped surface 9A. The lid 3, including the grinding tool 10 is then placed on the vessel 2 with the grinding tool 10 in the chamber 7. The lid 3 is rotated so that it engages the vessel 2. The shaft 20 is then connected to a power source, such as a drill commonly used in operating rooms, and the shaft 20 is rotated, thereby rotating the blade support 5A and the cutting blade 6. The cutting blade 6 rotates as the grinding tool 10 is advanced axially against the bone pieces on the bowl gripping plate 9B. As the bone pieces are ground or cut up, the particles pass through the rasp openings in the cutting blade 6 and are stored between the cutting blade 6 and the lid 3 within the chamber 7.

When axial resistance is felt from the grinding tool 10 coming into contact with the cup shaped surface 9A and bowl gripping plate 9B, the drill is disconnected from the shaft 20, and the lid 3 is removed from the vessel 2. The blade retainer 5 is then disconnected from the shaft 20 and the cutting blade 6 removed and discarded after the bone particles have been retrieved. The bone particles with blood and some tissue will remain in a glob and can be easily removed from the vessel 2.

The bone mill is then cleaned and after sterilization, a new cutting blade 6 is placed on the blade support 5A.

The above-described embodiments of the invention are intended to be examples only. Variations, alterations and modifications can be made to the particular embodiments described herein by those of skill in the art without departing from the scope of the invention, as defined in the appended claims.

1. A bone mill comprising a vessel and a rotatable grinding tool to produce ground autologous or allograft bone particles, wherein the vessel includes a wall defining a cylindrical chamber having a concentric axis, the vessel further includes a bottom wall which is complimentary to the shape of the cutting blade, a lid adapted to be removably connected to the vessel, the lid including an opening coincident with the axis, the grinding tool including an elongated shaft extending through the opening in the lid along the axis, a cutting blade mounted to the shaft within the chamber whereby the cutting blade can be rotated by motive means engaging the shaft exterior of the vessel to cut bone placed in the vessel into said bone particles.

2. A bone mill comprising a vessel and a rotatable grinding tool to produce ground autologous or allograft bone particles, wherein the vessel includes a wall defining a circular cylindrical chamber having a concentric axis, the vessel further includes a generally cup shaped bottom wall which corresponds to the shape of the cutting blade, a lid adapted to be removably connected to the vessel, the lid including an opening coincident with the axis, the grinding tool including an elongated shaft extending through the opening in the lid along the axis, the lid including bearing means for the shaft of the grinding tool to permit the shaft to rotate on the axis, a cutting blade mounted to the shaft within the chamber and a bone gripping bowl on the cup shaped bottom wall whereby the cutting blade can be rotated by motive means engaging the shaft exterior of the vessel to cut bone, placed in the vessel against the bone gripping bowl, into said bone particles.

3. A bone mill as defined in claim 2, wherein the cutting blade is a thin circular cup shaped blade with rasp openings and cutting edges, secured axially by the removable blade attachment to the end of the blade support member within the vessel, whereby the blade is rotated for cutting bone against the gripping bowl whereby bone particles pass through the rasp openings into the portion of the chamber between the cutting blade and the lid.

4. A bone mill as defined in claim 3, wherein the cup shaped cutting blade is a one-use blade removably attached to the blade support.

5. A bone mill as defined in claim 4, wherein the blade support includes a hub mounted for rotation at the end of the shaft within the vessel, and four spokes extend from the center of the hub to the periphery where the hub external part engages the blade to provide axial and rotational support to the blade.

6. A bone mill as defined in claim 4, wherein the means for a removable blade attachment of the cup shaped cutting blade to the end of the shaft is a fastener having a head and a shank, whereby the shank is adapted to engage the blade support member, and the head has an axial dimension which corresponds to the minimum spacing between the cutting blade and the gripping bowl during the operation of the bone mill.

7. A bone mill as defined in claim 2, wherein the bone gripping bowl is a hard metal disc that includes elongated, radially uninterrupted ridges placed at the bottom of the vessel, pressed fitted in the external sleeve.

8. A bone mill as defined in claim 2, wherein the shaft includes an end portion exterior of the vessel adapted to be engaged by a drill commonly used in an operating room providing rotation to the shaft and therefore the cutting blade.

9. A bone mill as defined in claim 8, wherein the shaft is rotated at a velocity between 500 and 3,000 rpm with up to 100 newton/m of torque.

10. A bone mill as defined in claim 2, wherein the lid includes a bearing portion co-extensive with the axis in the vessel for providing stability to the shaft while allowing low friction rotation to the shaft.
